(19) United States
(12) Patent Application Publication
(10) Pub. No.: US 2008/0133026 A1
(43) Pub. Date: Jun. 5, 2008

(54) ARTIFICIAL BONE AND A METHOD FOR MAKING THE SAME

(76) Inventors: Sio-Hou LEI, Kaohsiung City (TW); Kai-Lu WANG, Kaohsiung City (TW); Barry CHEN, Kaohsiung City (TW); Wenny KAO, Taipei Hsien (TW)

Correspondence Address: OSTROLENK FABER GERB & SOFFEN, 1180 AVENUE OF THE AMERICAS, NEW YORK, NY, 100368403, US

(21) Appl. No.: 11/551,298

(22) Filed: Oct. 20, 2006

(51) Int. Cl. A61F 2/36; B22D 19/14

(52) U.S. Cl. 623/23.54; 623/23.34; 623/901; 164/112

(57) ABSTRACT

An artificial bone includes a bone-shaped hollow metal body confining a sealed space and having two opposite closed ends, and a porous structure disposed in the sealed space. A method for making the artificial bone includes the steps of: positioning a metal tube in a bone-shaped mold cavity of a mold, hydro-forming the metal tube into a bone-shaped hollow tube in the bone-shaped mold cavity by injecting a high pressure fluid into the metal tube, disposing a crumple of metal wire into the bone-shaped hollow tube, depositing a metal coating on the metal wire and an inner surface of the bone-shaped hollow tube to form a porous structure in the bone-shaped hollow tube, and sealing the bone-shaped hollow tube.

### BACKGROUND OF THE INVENTION

1. Field of the Invention

The invention relates to an artificial bone, more particularly to an artificial bone having a porous structure. The invention also relates a method for making the artificial bone.

2. Description of the Related Art

Referring to FIG. 1, in a human skeleton, a neck 11 of each of femurs 1 is jointed to a lower side of a pelvis 2 to form a femoral joint, which provides walking and swinging functions, and which supports an upper body weight. The neck 11 of each of the femurs 1 is liable to fracture or to crash due to an impact occurred in, for example, a car accident or a fall. Additionally, bones of an elder may become brittle due to the loss of ossein therein, and thus fracture easily. When the aforesaid cases occur, it is required to replace the damaged bone with an artificial bone 3.

The artificial bone 3 used presently in the art is solid, and includes a bone body 31 inserted into the femur 1, and a neck portion 32 connected to the bone body 31 and bent relative to the bone body 31.

The method for making the artificial bone 3 includes the steps of: preparing a solid cylindrical rod of titanium alloy having a proper length; lubricating the surface of the rod; bending the rod by forging to form a preformed blank; cleaning and lubricating the surface of the preformed blank; forging the preformed blank at least two more times at a constant temperature to form a semi-product; and trimming the semi-product to obtain the artificial bone 3. It should be noted that the artificial bone 3 is required to be further processed, such as by rubbing, turning and localized-coarsening before being used in a human body.

In view of the aforesaid, the artificial bone 3 used presently in the art has the following disadvantages:

1. Since the raw material used for making the artificial bone 3 is a solid titanium alloy rod, it is necessary to use a relatively large-sized forging machine to produce a relatively large mechanical force for the forging process.

2. Since the artificial bone 3 is made of a solid titanium alloy rod, it is necessary to use a relatively large amount of raw material, and a relatively large amount of waste material is produced. Therefore, the production cost is relatively high.

3. Since the conventional method for making the artificial bone 3 involves various processing steps, such as lubricating, bending, cleaning, forging, etc., it is relatively complicated and time-wasting.

### SUMMARY OF THE INVENTION

One object of the present invention is to provide an artificial bone which is lightweight, and which can simulate the porous structure of a real bone.

Another object of the present invention is to provide a method for making the artificial bone, which reduces the amount of waste material, which is simple, and which does not require a large-sized processing machine.

Accordingly, in one aspect of this invention, an artificial bone includes a bone-shaped hollow metal body confining a sealed space and having two opposite closed ends, and a porous structure disposed in the sealed space.

In another aspect of this invention, a method for making an artificial bone includes the steps of: positioning a metal tube in a bone-shaped mold cavity of a mold, hydro-forming the metal tube into a bone-shaped hollow tube in the bone-shaped mold cavity by injecting a high pressure fluid into the metal tube, disposing a crumple of metal wire into the bone-shaped hollow tube, depositing a metal coating on the metal wire and an inner surface of the bone-shaped hollow tube to form a porous structure in the bone-shaped hollow tube, and sealing the bone-shaped hollow tube.

### BRIEF DESCRIPTION OF THE DRAWINGS

Other features and advantages of the present invention will become apparent in the following detailed description of the preferred embodiments with reference to the accompanying drawings, of which:

FIG. 1 is a schematic view showing a conventional artificial bone installed on a human skeleton;

FIG. 2 is a schematic sectional view of the preferred embodiment of an artificial bone according to this invention;

FIG. 3 is a flowchart of the preferred embodiment of a method for making an artificial bone according to this invention;

FIGS. 4 and 5 are schematic views showing consecutive steps of the method of the preferred embodiment;

FIG. 6 is an exploded perspective view showing a hydro-forming step of the method of the preferred embodiment; and

FIG. 7 is a schematic view showing an electroforming step of the method of the preferred embodiment.

### DETAILED DESCRIPTION OF THE PREFERRED EMBODIMENTS

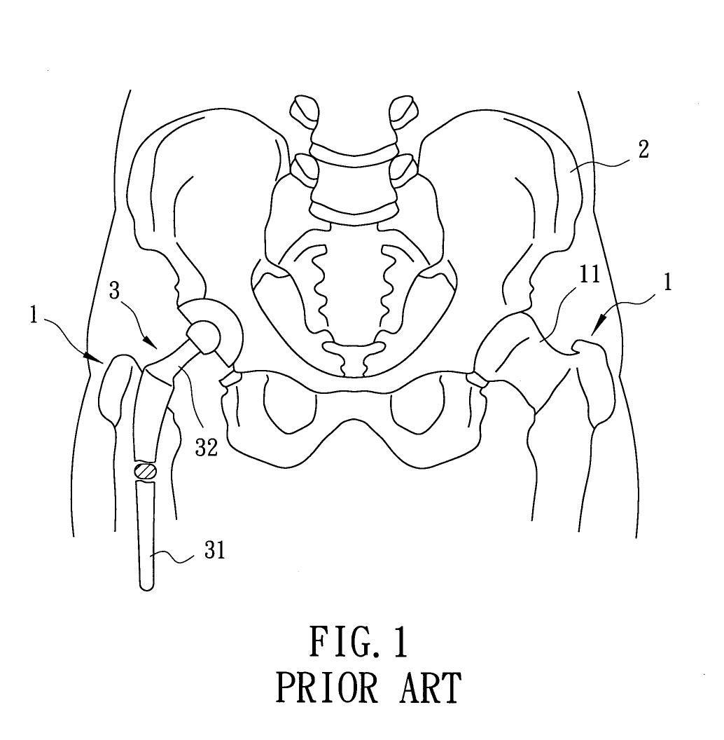

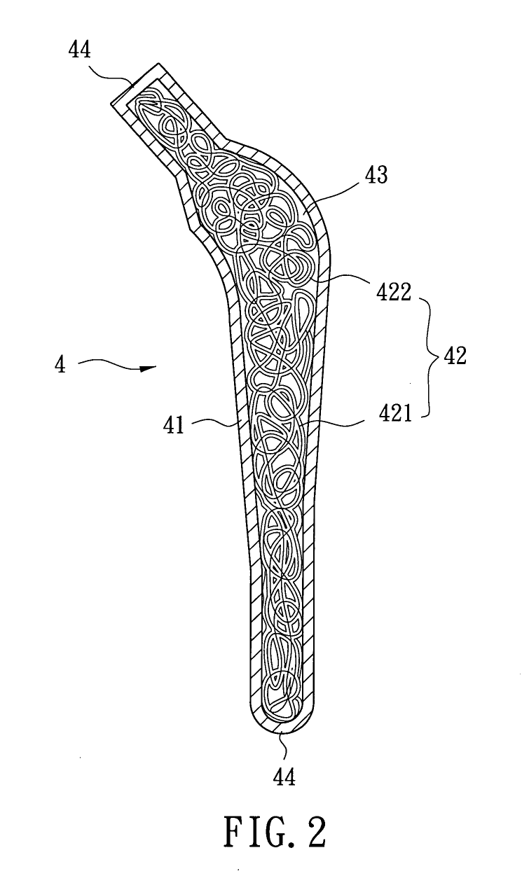

Referring to FIG. 2, the preferred embodiment of an artificial bone 4 according to this invention is shown to include a bone-shaped hollow metal body 41 confining a sealed space 43 and having two opposite closed ends 44, and a porous structure 42 disposed in the sealed space 43. In this preferred embodiment, the bone-shaped hollow metal body 41 is made of a titanium alloy. One of the closed ends 44 is dome-shaped for convenient installation of the artificial bone 4 into the femur 1 shown in FIG. 1. In practical use, the bone-shaped hollow metal body 41 is formed with a bent angle ranging from 120° to 140°. The actual angle depends on the specific requirement of a patient.

The porous structure 42 is disposed and fixed in the sealed space 43 of the bone-shaped hollow metal body 41, and includes a crumple of metal wire 421 and a metal coating 422 deposited on the metal wire 421 and an inner surface of the bone-shaped hollow metal body 41. In this preferred embodiment, the metal wire 421 is made of a titanium alloy. Alternatively, the metal wire 421 can be made of other suitable metals, such as a nickel alloy, a copper alloy, a gold alloy, a silver alloy, or the like. Furthermore, the crumple of metal wire 421 can be composed of one or more metal segments.

The metal coating 422 is deposited on the metal wire 421 and the inner surface of the bone-shaped hollow metal body 41 so as to form the porous structure 42 which simulates the sponge structure of a real bone, and which is fixed in the bone-shaped hollow metal body 41. In this preferred embodiment, the metal coating 422 is made of a titanium alloy. Alternatively, the metal coating 422 can be made of other suitable metals, such as a nickel alloy, a copper alloy, a gold alloy, a silver alloy, or the like.

It should be noted that since the bone-shaped hollow metal body 41 will come into contact with human cells, the material for the bone-shaped hollow metal body 41 should have superior human affinity. However, since the metal wire 421 and the metal coating 422 are disposed in the bone-shaped hollow metal body 41, various materials can be used therefor.

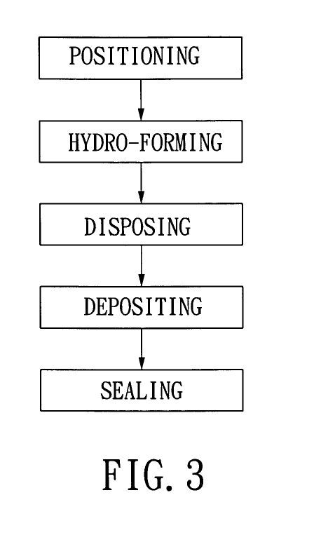

Referring to FIG. 3, the preferred embodiment of a method for making the artificial bone 4 according to this invention is shown to include the steps of:

A) Positioning:

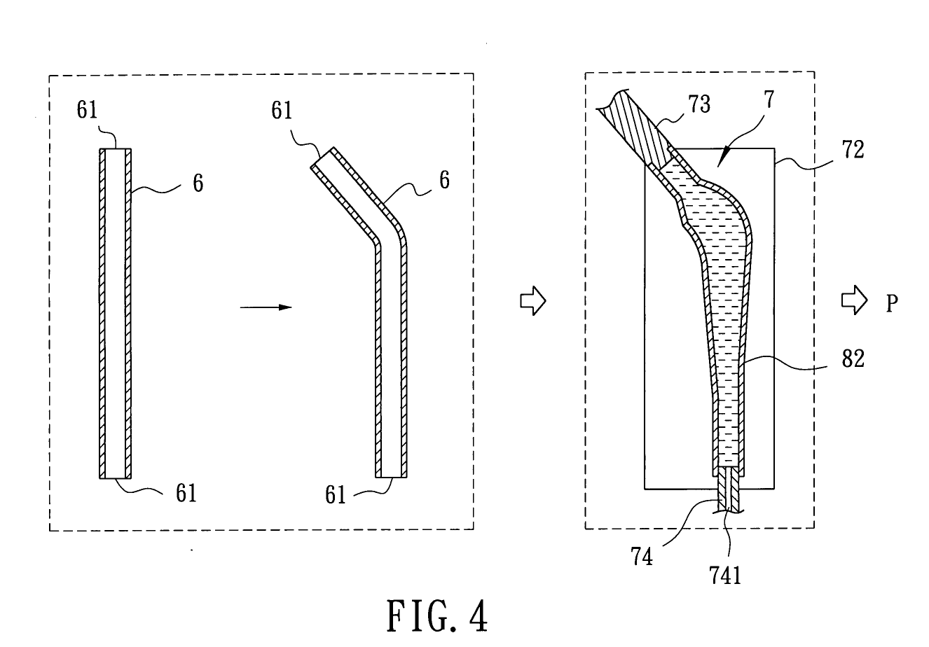

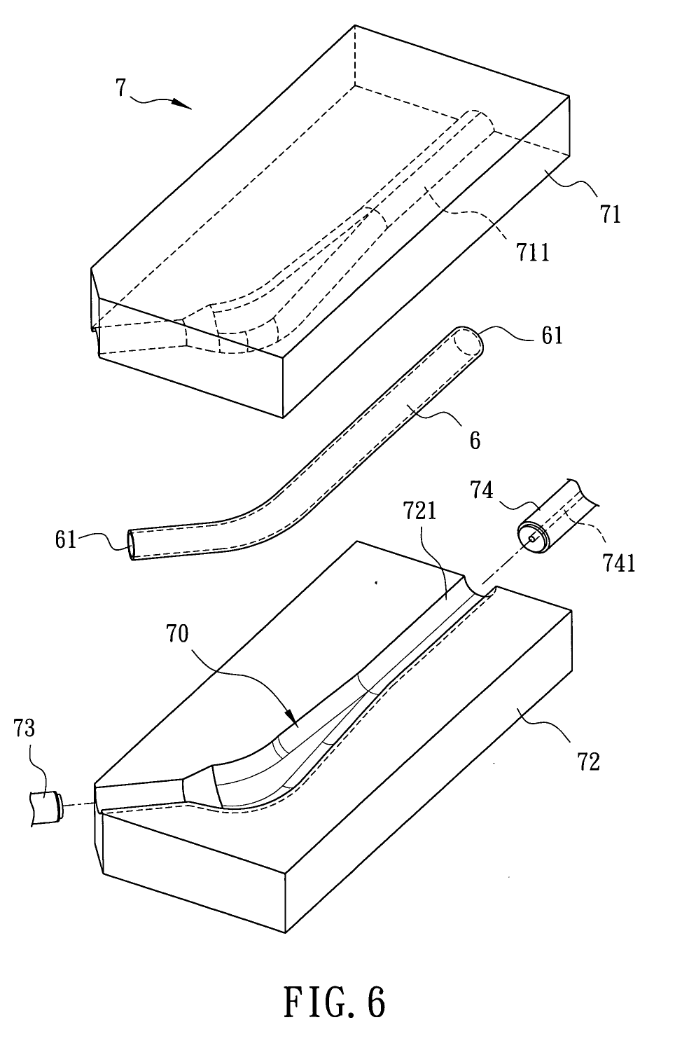

Referring to FIGS. 4 and 6, a metal tube 6 is bent and positioned in a bone-shaped mold cavity 70 of a mold 7. The tube 6 has two opposite open ends 61. In this preferred embodiment, the metal tube 6 is made of a titanium alloy, and is formed with a bent angle ranging from 120′ to 140°. The mold 7 includes an upper mold part 71 formed with an upper mold cavity portion 711, a lower mold part 72 formed with a lower mold cavity portion 721 cooperating with the upper mold cavity portion 711 to define the bone-shaped mold cavity 70, a first sealing block 73, and a second sealing block 74 having a fluid passage 741.

B) Hydro-Forming:

The mold 7 is closed by disposing the upper mold part 71 on the lower mold part 72 after the metal tube 6 is positioned in the lower mold cavity portion 721. The first sealing block 73 is sleeved on one of the open ends 61 of the metal tube 6 so as to close said one of the open ends 61. The second sealing block 74 is sleeved on the other one of the open ends 61 of the metal tube 6. The metal tube 6 is hydro-formed into a bone-shaped hollow tube 82 in the bone-shaped mold cavity 70 by injecting a high pressure fluid into the metal tube 6 via the fluid passage 741 of the second sealing block 74. The mold 7 is opened after releasing the fluid, and the bone-shaped hollow tube 82 is taken out of the mold 7.

C) Disposing:

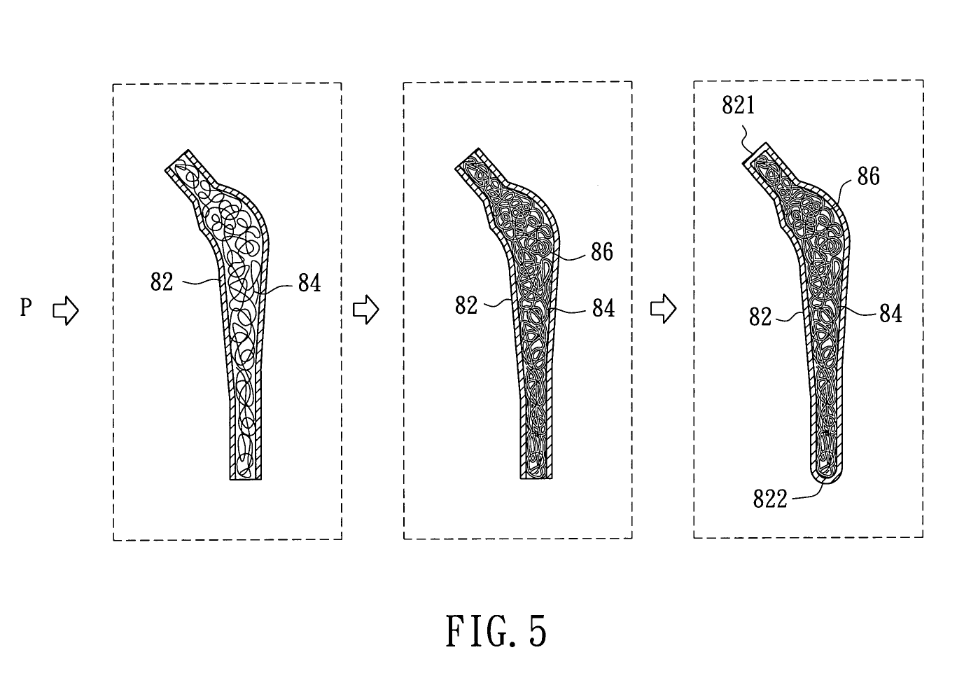

Referring to FIG. 5, a crumple of metal wire 84 is squeezed and disposed into the bone-shaped hollow tube 82 using an elongate bar having a cross section smaller than that of the bone-shaped hollow tube 82. As mentioned herein, the metal wire 84 used in this preferred embodiment is made of a titanium alloy, and other suitable metals, such as a nickel alloy, a copper alloy, a gold alloy, a silver alloy, or the like, can be used for the metal wire 84.

D) Depositing:

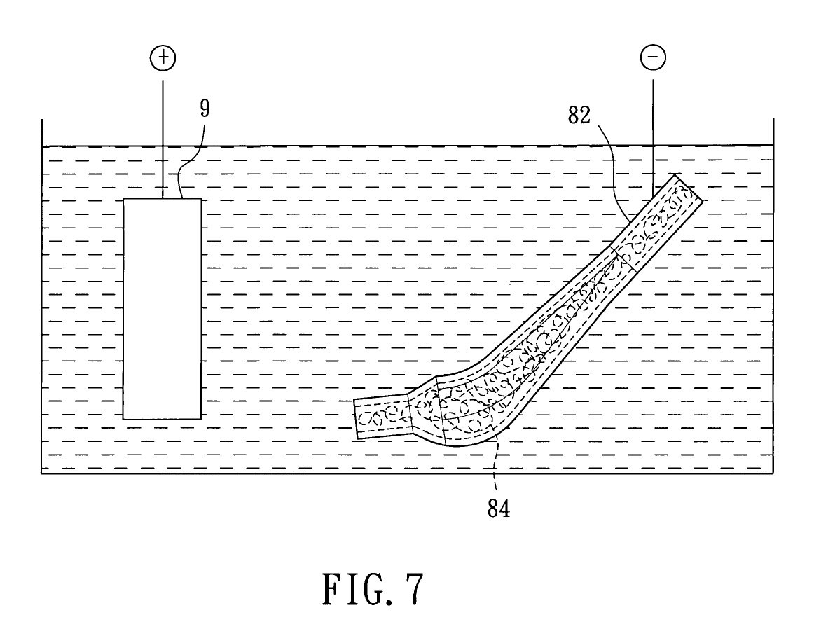

Referring to FIGS. 5 and 7, a metal coating 86 is deposited on the metal wire 84 and an inner surface of the bone-shaped hollow tube 82 by electroforming to form a porous structure fixed in the bone-shaped hollow tube 82. The electroforming is conducted by applying a non-conductive layer (not shown, such as releasable glue) on an outer surface of the bone-shaped hollow tube 82, electroforming the bone-shaped hollow tube 82 including the metal wire 84 in an electroforming medium, and removing the non-conductive layer from the outer surface of the bone-shaped hollow tube 82. The thickness of the metal coating 86 can be controlled by adjusting parameters, such as current density, electroforming time, etc.

In this preferred embodiment, the bone-shaped hollow tube 82 is connected electrically to a cathode, and a titanium alloy plate 9 is connected electrically to an anode. When an electric current is applied, the titanium alloy plate 9 dissociates to form metal ions, which migrate into the bone-shaped hollow tube 82 via the electroforming medium and form the metal coating 86 deposited on the metal wire 84 and the inner surface of the bone-shaped hollow tube 82.

E) Sealing:

Referring again to FIGS. 4 and 5, the open ends 61 of the bone-shaped hollow tube 82 are sealed by welding or the like to form an upper sealed end 821 and a lower sealed end 822 so as to obtain the artificial bone 4. The lower sealed end 822 is formed to be dome-shaped. Further processing, such as rubbing, turning and localized-coarsening, is performed before the artificial bone 4 is installed in a human body.

In addition to electroforming, other suitable methods can be used to form the porous structure. For example, sintered metal particles can be used to form the porous structure. Additionally, a removable material, such as a material having a low melting point, is disposed in the bone-shaped hollow tube 82, and a desirable metal is deposited on the removable material. The porous structure is formed after melting and removing the molten removable material from the bone-shaped hollow tube 82.

In view of the aforesaid, this invention has the following advantages:

1. Since the metal tube 6 used for forming the bone-shaped hollow tube 82 is hollow, the mechanical force required to perform the hydro-forming process is relatively small as compared to the prior art, and the raw material requirement in this invention is reduced significantly. Therefore, the production cost is lowered.

2. The processing step, such as forging, trimming, etc., required in the prior art can be omitted in this invention, and the method of this invention can be performed at ambient temperature. Therefore, the method of this invention is relatively simple.

3. The porous structure 42 formed in the artificial bone 4 of this invention can reduce the overall weight of the artificial bone 4 while providing a satisfactory mechanical strength.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

We claim:

1. An artificial bone, comprising:
a bone-shaped hollow metal body confining a sealed space and having two opposite closed ends; and
a porous structure disposed in said sealed space.

2. The artificial bone as claimed in claim 1, wherein said porous structure includes a crumple of metal wire and a metal coating deposited on said metal wire and an inner surface of said bone-shaped hollow metal body.

3. The artificial bone as claimed in claim 1, wherein said bone-shaped hollow metal body is made of a titanium alloy.

4. The artificial bone as claimed in claim 1, wherein said metal wire is made of a material selected from the group consisting of a titanium alloy, a nickel alloy, a copper alloy, a gold alloy, and a silver alloy.

5. The artificial bone as claimed in claim 1, wherein said metal coating is made of a material selected from the group consisting of a titanium alloy, a nickel alloy, a copper alloy, a gold alloy, and a silver alloy.

6. A method for making an artificial bone, comprising the steps of:
positioning a metal tube in a bone-shaped mold cavity of a mold;
hydro-forming the metal tube into a bone-shaped hollow tube in the bone-shaped mold cavity by injecting a high pressure fluid into the metal tube;
disposing a crumple of metal wire into the bone-shaped hollow tube;
depositing a metal coating on the metal wire and an inner surface of the bone-shaped hollow tube to form a porous structure in the bone-shaped hollow tube; and
sealing the bone-shaped hollow tube.

7. The method as claimed in claim 6, further comprising a step of bending the metal tube before placing in the mold cavity.

8. The method as claimed in claim 6, wherein the metal wire is squeezed into the bone-shaped hollow tube using an elongate bar having a cross section smaller than that of the bone-shaped hollow tube.

9. The method as claimed in claim 6, wherein the depositing step is conducted by electroforming.

10. The method as claimed in claim 9, wherein the electroforming is conducted by applying a non-conductive layer on an outer surface of the bone-shaped hollow tube, electroforming the bone-shaped hollow tube including the metal wire in an electroforming medium, and removing the non-conductive layer from the outer surface of the bone-shaped hollow tube.

11. A method for making an artificial bone, comprising the steps of:
forming a bone-shaped hollow metal tube;
disposing a metallic porous structure in the hollow metal tube; and
sealing the hollow metal tube.
